(19) United States
(12) Patent Application Publication
(10) Pub. No.: US 2021/0139832 A1
(43) Pub. Date: May 13, 2021

(54) METHOD FOR PRODUCING AT LEAST ONE CLOSED REGION ON A CARRIER SURFACE OF A CARRIER

(71) Applicant: cytena GmbH, Freiburg (DE)

(72) Inventors: Jonas SCHÖNDUBE, Freiburg (DE); Ludwig GUTZWEILER, Freiburg (DE); Julian RIBA, Freiburg (DE); Andre GROSS, Freiburg (DE); David BANCROFT, Gauting (DE)

(21) Appl. No.: 17/045,810

(22) PCT Filed: Apr. 9, 2019
(86) PCT No.: PCT/EP2019/058896

§ 371 Date: Oct. 7, 2020

(30) Foreign Application Priority Data
Apr. 9, 2018 (LU) 100772

(51) Int. Cl. C12M 1/12; C12M 1/22

(52) U.S. Cl. CPC C12M 25/00; C12M 23/10

(57) ABSTRACT

The invention relates to a method for producing at least one closed region on a carrier surface of a carrier, wherein the method comprises the following steps:
a. adding a first fluid comprising at least one cell and/or at least one particle to the carrier surface and
b. adding a second fluid, wherein the second fluid is immiscible with the first fluid and at least partially covers the first fluid and
c. acquiring at least one item of cell information and/or of at least one item of particle information and
d. producing the closed region on the basis of the at least one acquired item of cell information and/or the at least one acquired item of particle information.

### CROSS-REFERENCE TO RELATED APPLICATION

The present application is the U.S. national phase of International Application No. PCT/EP2019/058896 filed Apr. 9, 2019, which claims the benefit of and priority to Luxembourgian Patent Application No. 100772 filed Apr. 9, 2018, the entire disclosure of which is incorporated herein by reference.

### FIELD

The disclosure relates to a method for producing at least one closed region on a carrier surface of a carrier. The disclosure also relates to a device for producing at least one closed region on a carrier surface of a carrier. The disclosure also relates to a system having such a device and the carrier.

### BACKGROUND

It is known from the prior art that active substances, such as monoclonal antibodies and other proteins, are produced with the aid of so-called monoclonal cell lines. These are populations of cells that are all descended from a single parent cell. The production of monoclonal cell lines is necessary because this is the only way to ensure that all cells of the population have approximately the same genome in order to produce the active ingredients with constant and reproducible quality.

In order to produce a monoclonal cell line, cells are transferred individually into the containers of a microtitre plate. The cells to be transferred are produced by genetically modifying a host cell line and isolating these modified cells. Individual cells are deposited in the microtitre plates using, for example, free jet printing methods or pipetting. After the cells have been deposited in the respective containers of the microtitre plate, the cells can grow and may then be transferred to a bioreactor.

A device from the company iota Sciences Ltd. is known from the prior art, in which the cells are not deposited in containers of the microtitre plate, but instead in a Petri dish. Before the cells are deposited in the Petri dish, the Petri dish is placed in a receptacle of the device. The Petri dish contains two liquids that are immiscible with one another, wherein the second liquid is added to the Petri dish after the first liquid and completely covers the first liquid. The second liquid can be an oil such as FC-40.

A force is exerted on part of the two liquids by means of a hydrophobic pin, so that the part of the second liquid wets the bottom of the dish. In particular, the pin is moved in such a way that the part of the second liquid wetting the base of the dish forms a grid-like pattern. As a result, the part of the second liquid separates a plurality of regions each having the first liquid from one another.

After the grid-like pattern has been created, a cell suspension is applied to each of the regions of the first liquid, wherein the cell suspension applied to the respective region can contain a cell. The user then manually checks for each region, for example by means of a microscope, whether the cell suspension applied has a cell in each region. If this is the case, the region containing the cell is marked manually by the user.

The aforementioned device has the disadvantage that a large number of time-consuming work steps are required in order to determine the regions containing the cell.

### SUMMARY

The object of the disclosure is therefore to provide a method that enables more efficient workflows in the laboratory.

The object is achieved by a method for producing at least one closed region on a carrier surface of a carrier, wherein the method comprises the following steps:

a. adding a first fluid comprising at least one cell and/or at least one particle to the carrier surface and

b. adding a second fluid, wherein the second fluid is immiscible with the first fluid and at least partially covers the first fluid and

c. acquiring at least one item of cell information and/or of at least one item of particle information and

d. producing the closed region on the basis of the at least one acquired item of cell information and/or the at least one acquired item of particle information.

In addition, the object of the disclosure is to disclose a device by means of which work processes in the laboratory can be carried out more efficiently.

The object is achieved by a device for producing at least one closed region on a carrier surface of a carrier for receiving a first fluid and a second fluid, wherein the second fluid is immiscible with the first fluid and at least partially covers the first fluid, having an acquisition device for acquiring at least one item of cell information and/or of at least one item of particle information and a displacement agent, by means of which, on the basis of the at least one acquired item of cell information and/or the at least one acquired item of particle information, the closed region can be produced.

The solution according to the disclosure has the advantage that at least one item of cell information and/or item of particle information is acquired. The item of cell information and/or item of particle information can contain information about the position of the cell and/or the particle in the first fluid and/or the morphology, such as the size and/or roundness, of the cell and/or the particle and/or about the optical properties of the cell and/or the particle. The optical properties can relate to the contrast, the fluorescence and/or the granularity of the cell and/or the particle. By evaluating the item of cell information and/or item of particle information, cells or particles relevant to the user can be identified in a simple manner and enclosed in a region.

Another advantage of the disclosure is that three fluids are no longer added, but instead only the first fluid and the second fluid. As a result, the method according to the disclosure is simplified compared to the methods known from the prior art.

It is also advantageous if the first fluid containing the cells and/or particles is applied to the carrier surface of the carrier not belonging to the device before the region or regions are produced. This is advantageous because the time-consuming generation of the grid-shaped pattern is no longer necessary. It is therefore not necessary to produce a large number of regions, but rather, as described in detail below, certain regions, for example those of interest to the user, can be produced. The cells and/or particles contained in the individual regions can undergo further processing steps.

The above process steps a to d can be carried out in the order stated.

The first fluid can be an, in particular aqueous, liquid and/or have a semi-solid consistency. In particular, the first fluid can be a cell suspension which promotes growth of the cells located in the first fluid. The first fluid can have agar, a semi-solid medium or gel in order to have a firmer consistency. The second fluid can be a liquid. Preferably, the second fluid can be an oil, such as a fluorine-inert (fluorocarbon-based) liquid, which is immiscible with the first fluid. In particular, the second fluid can be FC-40. Due to the immiscibility of the second fluid with the first fluid, the first fluid and the second fluid are arranged separately from one another and/or the second fluid is arranged on the first fluid. The second fluid can partially or completely cover the first fluid, in particular the surface of the first fluid facing the second fluid. The carrier can be a container such as a Petri dish. The carrier surface can be a container base. Alternatively, the carrier can be a plate without a side wall.

The part of the second fluid that wets the carrier surface serves to fluidically separate the closed region from a remaining section of the first fluid. The wetting of the carrier surface with the part of the second fluid takes place in such a way that, after wetting the part of the second fluid wetting the carrier surface, this is not pressed back into its starting position by the first fluid. The starting position is understood to be the position of the second fluid in which the second fluid does not wet the carrier surface.

A closed region is understood to be a region on the carrier surface that is fluidically separated from the remaining section of the first fluid. In particular, the first fluid located in the region is fluidically separated from the first fluid located in the remaining section. The separation can, as described above, take place at least partially through the part of the second fluid. Thus, in addition to the second fluid, the closed region can be delimited by at least one side wall of the carrier and/or the carrier surface.

The closed region can have at least one cell and/or at least one particle. However, it is alternatively possible for the region to have a predefined number or no cells and/or no particles. This region is referred to below as the remaining region.

In a particular embodiment, the region can be produced in that the first fluid is displaced in such a way that part of the second fluid wets the carrier surface. This makes it easy to produce the region. The first fluid can be displaced by a displacement agent. Alternatively or additionally, the second fluid can be displaced by the displacement agent. To displace the first and/or second fluid, the displacement agent and/or the carrier and thus the carrier surface can be moved.

The part of the second fluid that wets the carrier surface can form a closed line. This creates a closed region which is separated in a simple manner by the part of the second fluid from the remaining section of the first fluid. In addition, in this case the closed region is only limited by the second fluid and the carrier surface.

The closed region can have a very small volume, for example a volume between 0.5 nl (nanolitres) and 10 μl (microlitres). Regions with such a small volume enable cell growth to take place in certain cells. This arises because some cells can only grow if their concentration in the first fluid is not too low. The cells themselves have a smaller volume than the region. Likewise, the particles have a smaller volume than the region, wherein the particles can be glass or polymer beads which are introduced into the first fluid.

Using the method according to the disclosure and/or the device according to the disclosure, a plurality of regions can be produced. This is useful when a plurality of cells are arranged in the first fluid. In this case, the plurality of regions are produced in such a way that each of the plurality of regions has at least one, in particular precisely one, single cell and/or at least one particle, in particular precisely one single particle. The plurality of regions can be arranged adjacent to one another, that is to say separated from one another only by the part of the second fluid which wets the carrier surface. Alternatively, the plurality of regions can be separated from one another by a remaining region which has the first fluid and which does not contain any cells or particles. In this case, the separation between the remaining region and the plurality of regions also takes place by means of the part of the second fluid that wets the carrier surface.

The plurality of regions can have different cross-sections. In particular, the plurality of regions can differ from one another in the shape and/or the size of the cross-sections. This offers the advantage that a region that matches the cell can be produced. This is necessary because, as previously described, different cells require different volumes, for example, in order to be able to grow. Alternatively, the plurality of regions can have the same cross-section. The cross-section corresponds to a plane having the region and running parallel to the carrier surface. In addition, the plurality of regions can differ from one another in their formation along a normal to the carrier surface.

In a particular embodiment, on the basis of the item of cell information and/or the item of particle information, it can be determined where the at least one region is to be produced. It is particularly advantageous if the position of the at least one cell or of the particle in the first fluid is determined as an item of cell information or item of particle information before the region is produced. This offers the advantage that it is easily known at which points on the carrier the regions are to be produced. In particular, it is no longer necessary for a grid-shaped pattern to be produced before the first fluid is added to the carrier surface.

Alternatively or additionally, cells and/or particles that are particularly relevant can be selected on the basis of the item of cell information and/or item of particle information and only the selected cells or particles are enclosed by means of the part of the second fluid and/or only regions are produced which contain the selected cells and/or particles.

The item of cell information and/or item of particle information can be determined automatically, for example by means of the optical acquisition device described below. This reduces the workload for the user of the device because they no longer have to manually determine the position of the cells or particles themselves, for example.

To acquire the item of cell information and/or item of particle information, the device can have the optical acquisition device. The acquisition device can have an optical imaging device. The imaging device can produce an image of the carrier, in particular the carrier surface. The imaging device can, for example, be a camera with optics, for example similar to a microscope.

In addition, the device can have an evaluation device which determines the at least one item of cell information and/or at least one item of particle information on the basis of the image. In particular, the position of the cell and/or the particle in the first fluid can be determined by means of the evaluation device. The position of the cell and/or the particle can be determined by defining at least one optical property, in particular from the image and/or the cell and/or the particle, by means of the evaluation device. The evaluation device can be electrically connected to a control device. The control device can control a moving device in such a way that the moving device moves the displacement agent and/or the carrier in order to produce the region.

The device can have a receptacle for receiving the carrier, wherein the imaging device and the displacement agent are mutually opposite one another with respect to the receptacle. In particular, the displacement agent can be arranged above the carrier surface and the imaging device can be arranged below the carrier surface. As a result, a compact and simply constructed device is realised.

Before the region is produced, the carrier can be moved on the basis of the acquired item of cell information and/or item of particle information. In particular, the carrier surface can be moved in such a way that the at least one cell and/or the at least one particle are moved into a new position. The carrier can be shaken or vibrated and/or the first liquid mixed to reposition the cell or particle in the first fluid. The carrier is preferably moved when it is determined by the evaluation device that some cells and/or particles are adhering to one another and/or are arranged too close to one another. As a result, a homogeneous distribution of the cells and/or particles in the first fluid should be achieved by moving the carrier.

After the region has been produced, the remaining region of the first fluid which has no cells can be removed. As a result, only the regions containing the cells and/or particles remain on the carrier surface, so that the user can immediately see in which regions of the carrier the cells and/or particles are located. For at least partial removal of the remaining regions, the device can have a removal device. The remaining region can be removed by aspirating the remaining regions and/or flushing away the remaining regions. In particular, the first fluid and the second fluid of the remaining region can be removed.

In a special embodiment, the region can be divided into at least two sub-regions. This is useful, for example, after cells have grown. By dividing the region, a part of the cells can be examined separately from the other part of the cells. The division of the region can take place in that the first fluid of the region is displaced in such a way that part of the second fluid of the region wets the carrier surface. The first fluid can be displaced by the displacement agent.

It is also advantageous if a reagent is entered in a region having a cell. This allows cell growth within the region to be promoted or stopped in a simple manner. In particular, reagents for analysing the cell or other components of the region can also be entered. The reagent can be added using a dispensing device. A compact device can be implemented if the removal device and the dispensing device are designed as one structural unit.

In addition, the region having a cell can be sucked out, in particular after a predetermined period of time. The sucked out cells can then be processed further, for example in a bioreactor or a microtitre plate. The at least partial suction can be carried out by the removal device. Alternatively, it is possible that a separate suction device is available.

In a particular embodiment, the displacement agent can have a solid body, in particular a hydrophobic solid body. The first fluid can be displaced by the solid body. When the first fluid is displaced, the solid body is in direct contact with the first fluid. The solid body can be designed in the shape of a pin or a rod. The hydrophobic design of the displacement agent offers the advantage that the first fluid does not adhere to the displacement agent, so that the second fluid can easily wet the carrier surface. The displacement agent can have a rounded end at its end facing towards the carrier surface.

To create the closed region, the displacement agent can be moved in at least one direction, in particular precisely two directions, wherein the direction is parallel to the carrier surface. The displacement agent can be moved in the direction parallel to the carrier surface after the displacement agent contacts the carrier surface directly. Alternatively, the displacement agent can be moved in the direction parallel to the carrier surface after the displacement agent has displaced the second fluid in such a way that it wets the carrier surface and is arranged between the displacement agent and the carrier surface. In this case the displacement agent is not in direct contact with the carrier surface.

Alternatively, the displacement agent can have an, in particular cylindrical, triangular or rectangular, pattern element at its end facing towards the first and second fluid. In particular, the pattern element can be designed to be hollow. The pattern element can be detachably connected to a remaining section of the displacement agent. This offers the advantage that the pattern element can be dismantled after use or replacement with another pattern element.

Depending on the design of the pattern element, it is possible, for example, to realise a region with an annular, triangular or rectangular cross-section. In addition, by means of differently designed pattern elements, regions can also be produced in a simple manner which are designed differently along the normal to the carrier surface and/or have different heights. The provision of the pattern element thus offers the advantage that the region can be produced very quickly. In particular, to produce the region, the displacement agent can be moved exclusively in the direction of the carrier surface. The displacement agent can only be moved vertically. Alternatively or additionally, the carrier can be moved in the direction of the displacement agent in order to produce the region. In particular, the carrier can only be moved vertically.

In addition, the cell or the particle can be completely enclosed by a displacement agent designed in this way after the displacement agent has been lowered by the part of the second fluid. This offers the advantage that the cell and/or the particle can be moved into a different position on the carrier surface by means of the displacement agent. For this purpose, the displacement agent and/or the carrier surface can be moved in at least one direction parallel to the carrier surface.

In an alternative embodiment, the displacement agent can have a gas outlet opening. The gas outlet opening can be arranged at the end of the displacement agent facing the first and second fluid. In this case, a gas can be output through the gas outlet opening, which acts on the first fluid and thus displaces the first fluid. By using the gas, it is avoided in a simple manner that a solid body of the displacement agent comes into direct contact with the first and/or second fluid.

A device which is suitable for carrying out the method according to the disclosure is particularly advantageous. In addition, a system is advantageous which has the device according to the disclosure and the carrier which receives the first fluid and the second fluid, wherein the second fluid is immiscible with the first fluid and at least partially covers the first fluid. The carrier can be received by the device.

### BRIEF DESCRIPTION OF THE DRAWING VIEWS

The subject of the disclosure is shown schematically in the figures, wherein elements that are the same or have the same effect are mostly provided with the same reference symbols. In the drawings:

FIG. 1 shows the carrier with a first fluid and a second fluid,

FIG. 2 shows a side view of part of the device according to the disclosure before a region is produced,

FIG. 3 shows a side view of part of the device according to the disclosure, in which a region having a cell is produced according to a first operating mode of the displacement agent,

FIG. 4 shows a side view of part of the device according to the disclosure, in which a region having a cell is produced according to a second operating mode of the displacement agent,

FIG. 5 shows a side view of part of the device according to the disclosure after a plurality of regions have been produced, each having a cell,

FIG. 6 shows a plan view of a carrier after a plurality of regions have been produced and

FIG. 7 shows a side view of part of the device according to the disclosure with a region having a cell.

### DETAILED DESCRIPTION

FIG. 1 shows a carrier 3 which is designed as a container. The carrier 3 contains a first fluid 4 and a second fluid 5 which is immiscible with the first fluid 4. The first fluid 4 contains a plurality of cells 1. Designs are also conceivable in which the first fluid 4 alternatively or additionally contains particles. The first fluid 4 and the second fluid 5 are added to the carrier 3. The first fluid 4 can preferably be added before the second fluid 5. The second fluid 5 is arranged on the first fluid 4 and completely covers this within the carrier 3.

FIG. 2 shows a side view of part of the device 10 according to the disclosure before a region is produced. As can be seen from FIG. 2, the carrier 3 is arranged on a receptacle 13 of the device 10. The device 10 has a displacement agent 6 which can be moved along the directions x, z, y. In addition, the device 10 has an optical acquisition device 25. The acquisition device has an imaging device 11, by means of which an optical image of the carrier 3, in particular a carrier surface 7, can be produced.

In addition, the acquisition device 25 has an evaluation device 12. The imaging device 11 is electrically connected to the evaluation device 12. The evaluation device 12 serves to determine items of cell information, such as the position of the cells 1 located in the first fluid 4 on the carrier surface 7. The evaluation device 12 can be electrically connected to a control device, not shown in detail. The imaging device 11 and the displacement agent 6 can lie opposite one another with respect to the receptacle 13 of the device 10 or can be arranged offset from one another.

The device 10 can have a moving device 26 which is controlled by the control device. The moving device 26 can move the acquisition device 25 and/or the displacement agent 6 and/or the carrier 3 along the directions x, y, z. In particular, the moving device 26 can move the displacement agent 6 and/or the carrier 3 on the basis of the item of cell information determined by the evaluation device 12 in order to produce a region described in more detail below.

The displacement agent 6 is designed as a hydrophobic pin. The displacement agent 6 has a rounded end at its end facing towards the second fluid 5.

FIG. 3 shows a side view of part of the device 10 according to the disclosure, in which a closed region 2 having a cell is produced according to a first operating mode of the displacement agent 6. Before the region 2 is produced, the item of cell information is determined by means of the acquisition device 25. In particular, the regions of the carrier surface 7 in which the cells 1 are arranged are determined.

The displacement agent 6 is then moved in such a way that it creates the closed region 2 which includes the cell 1. To produce the region 2, the displacement agent 6 is first moved along the direction y to the carrier surface 7. After wetting the carrier surface 7 by a part of the second fluid 5, the displacement agent 6 is moved along the directions z and x in order to produce the closed region 2. When the displacement agent 6 moves along the directions z and x, the first fluid 4 is also displaced by the displacement agent 6, so that the second fluid 5 can wet the carrier surface 7.

In this operating mode, the displacement agent 6 presses the part of the second fluid 5 in the direction of the carrier surface 7 until the part of the second fluid 5 wets the carrier surface 7. In this operating mode, the displacement agent 7 does not come into direct contact with the carrier surface 7. As can be seen from FIG. 4, the part of the second fluid 5 is arranged between the displacement agent 6 and the carrier surface 7.

The region 2 is fluidically separated from a remaining section 18 by means of the part of the second fluid 15, wherein the remaining section 18 has the first fluid 4 with the plurality of cells 1. The fluidic separation takes place such that the first fluid 4 located in the region 2 is not fluidically connected to the first fluid 4 located in the remaining section 18.

FIG. 4 shows a side view of part of the device 10 according to the disclosure, in which a closed region 2 having a cell 1 is produced according to a second operating mode of the displacement agent 6. In this operating mode, the displacement agent 6 is moved in such a way that, when it moves along the direction y, it passes through the second fluid 5 and displaces the first and second fluids 4, 5. The displacement agent 6 comes into direct contact with the carrier surface 7.

After the displacement agent 6 contacts the carrier surface 7, it is moved in the direction x and/or z in order to produce the closed region 2. When the displacement agent 6 is moved along the x and/or z direction, the first fluid 4 is also displaced. After the displacement agent 6 has moved, part of the second fluid 5 flows into the portion of the first fluid 4 displaced by the displacement agent 6 and thus wets the carrier surface 7.

FIG. 5 shows a side view of part of the device according to the disclosure after a plurality of regions 2 have been produced, each having a cell 1. In particular, FIG. 5 shows that precisely three regions 2, namely a first region 22, a second region 16 and a third region 17, were produced by means of the device 10. In addition, the carrier 3 contains a remaining region 8 that does not contain any cells.

The individual regions 2 are fluidically separated from one another and from the remaining region 8 by part of the second fluid 15. In particular, the first region 22 is fluidically separated from the remaining region 8 by a first part of the second fluid 23. The remaining region 8 is also fluidically separated from the second region 16 by means of a second part of the second fluid 19. The second region 16 is further fluidically separated from the third region 17 by means of a third part of the second fluid 20.

To produce the regions 2, as already described in FIG. 3, after the two fluids 4, 5 have been introduced into the carrier 3 by means of the imaging device 11, an image of the carrier 3, in particular the carrier surface 7, or, by means of image stacking, of the entire fluid volume is produced. The evaluation device 12 determines the item of cell information, in particular the position of the cells 1 located in the first fluid 4, on the basis of the produced image.

To produce the first region 22, the displacement agent 6 is moved on the basis of the determined positions of the cells 1 into a position relative to the carrier 3 and, as shown in FIG. 3 or 4, is lowered along the direction y in the direction of the carrier surface 7.

Here, the rounded end of the displacement agent 6 displaces the first fluid 4 in such a way that the first part of the second fluid 23 wets the carrier surface 7. The displacement agent 6 is then moved along the directions z, x, which run parallel to the carrier surface 7, to produce the first region 22.

The first region 22 is fluidically separated from the rest of the first fluid 4 located within the carrier 3 by means of the first part of the second fluid 23. The first region 22 is thus delimited by the carrier surface 7, a side wall of the carrier 3, the first part of the second fluid 23 wetting the carrier surface 7 and the second fluid 5 wetting the first region 22. After the first region 22 has been produced, the displacement agent 6 is moved away from the carrier surface 7 in the direction y. The displacement agent 6 is then moved into a different position relative to the carrier 3, starting from which the second region 16 shown in FIG. 5 is produced. After completion of the second region 16, the third region 17 is produced.

The second and third regions 16, 17 are produced analogously to the first region 22, in that the displacement agent 6 displaces the first fluid 4 in such a way that the corresponding part of the second fluid 19, 20 wets the carrier surface 7. In addition, the displacement agent 6 is moved parallel to the carrier surface 7. Even when the displacement agent 6 moves parallel to the carrier surface 7, the first fluid 4 is displaced in such a way that the corresponding part of the second fluid 19, 20 wets the carrier surface 7.

The device 10 also has a removal device 14. The remaining region 8 can be sucked out by means of the removal device 14. In this case, the removal device 14 serves to remove the remaining region 8 which does not have any cells. As an alternative or in addition, the removal device 14 can serve to suck out the regions 2. The suction takes place after a predetermined period of time or inspection, so that cell growth has taken place in the respective regions 2.

FIG. 6 shows a plan view of a carrier 3 after a plurality of regions 2, namely the first region 22, the second region 16 and the third region 17, have been produced. The arrangement of the cells 1 in the carrier 3 shown in FIG. 6 differs from the arrangement of the cells 1 in the carrier 3 shown in FIGS. 1 to 5.

As can be seen from FIG. 6, the three regions 2 differ in their cross-section. In addition, the three regions 2 differ from one another in their design and volume. The first region 22 has a circular shape, the second region 16 has a rectangular shape and the third region 17 has a triangular shape. The remaining region 8 was removed by means of the removal device 14.

The second region 16 can be divided into two sub-regions 9. This can occur by the displacement agent 6 displacing the first fluid 4 of the second region 16. As a result, a fourth part of the second fluid 21 wets the carrier surface 7. The fourth part of the second fluid 21 separating the two sub-regions 9 is shown in FIG. 6 by a dashed line.

The regions 2 shown in FIG. 6 can have been produced by means of a method shown in FIGS. 2-5. This means that the displacement agent 6, after it has already pressed the respective part of the second fluid 15 against the carrier surface 7 or is itself in direct contact with the carrier surface 7, is moved along the x, y direction. Alternatively, the regions shown in FIG. 6 can be produced according to the method described below in connection with FIG. 7.

FIG. 7 shows a side view of part of the device 10 according to the disclosure with a region 2 having a cell 1. The device 10 differs from the device 10 shown in FIG. 3 or FIG. 4 in the design of the displacement agent 6. Thus, the displacement agent 6 shown in FIG. 7 has a pattern element 27 at its end facing the carrier surface 7, by means of which, for example, a cross-section circular region 2 can be produced. The pattern element 27 is designed as a hollow cylinder.

To produce the closed region 2, the moving device 26 moves the displacement agent 6 exclusively in direction y. The displacement agent 6 thus presses the part of the second fluid 15 in the direction of the carrier surface 7 until the same wets the carrier surface 7. Alternatively, the displacement agent 6 can be operated in a similar way to the embodiment shown in FIG. 4, such that the displacement agent 6 displaces the first fluid 4 such that it comes into direct contact with the carrier surface 7. After the displacement agent 6 contacts the carrier surface 7, this is moved in the opposite direction in direction y. A part of the second fluid 5 wets the carrier surface 7, so that a fluidic separation between the region 2 and the remaining section 18 of the first fluid is realised.

By using the displacement agent 6 shown in FIG. 6, it is not necessary to move the displacement agent 6 in the directions x, z in order to produce the closed region 2. The region can namely be produced solely by moving the displacement agent 6 exclusively in direction y.

### LIST OF REFERENCE NUMBERS

1 Cell

2 Region

3 Carrier

4 First fluid

5 Second fluid

6 Displacement agent

7 Carrier surface

8 Remaining region having no cells

9 Sub-region

10 Device

11 Imaging device

12 Evaluation device

13 Receptacle

14 Removal device

15 Part of the second fluid

16 Second region

17 Third region

18 Remaining section

19 Second part of the second fluid

20 Third part of the second fluid

21 Fourth part of the second fluid

22 First region

23 First part of the second fluid

25 Acquisition device

26 Moving device

27 Pattern element

x, y, z Direction

1. A method for producing at least one closed region (2) on a carrier surface (7) of a carrier (3), wherein the method comprises the following steps:
a. adding a first fluid (4) comprising at least one cell (1) and/or at least one particle to the carrier surface (7) and
b. adding a second fluid (5), wherein the second fluid (5) is immiscible with the first fluid (4) and at least partially covers the first fluid (4) and
c. acquiring at least one item of cell information and/or of at least one item of particle information and
d. producing the closed region (2) on the basis of the at least one acquired item of cell information and/or the at least one acquired item of particle information.

2. The method according to claim 1, wherein
a. the region (2) is produced in that the first fluid (4) is displaced in such a way that part of the second fluid (15) wets the carrier surface (7) or
b. the region (2) is produced in that the first fluid (4) is displaced in such a way that part of the second fluid (15) wets the carrier surface (7) and the part of the second fluid (15) forms a closed line.

3. The method according to claim 1, wherein
a. a plurality of regions (2) are produced, wherein the regions (2) have the same or different cross-sections and/or
b. a plurality of regions (2) are produced which differ from one another in their formation along a normal to the carrier surface (7).

4. The method according to claim 1, wherein
a. on the basis of the item of cell information and/or the item of particle information, it is determined where the at least one region (2) is produced and/or
b. the region (2) has at least one cell (1) and/or at least one particle.

5. The method according to claim 1, wherein before the region (2) is produced
a. on the basis of the acquired item of cell information and/or item of particle information, the carrier (3) is moved or
b. on the basis of the acquired item of cell information and/or item of particle information, the carrier (3) is moved in such a way that the at least one cell (1) and/or the at least one particle is moved into a new position or
c. on the basis of the acquired item of cell information, the carrier is shaken or vibrated and/or the first fluid is mixed so that the at least one cell (1) and/or the at least one particle is moved into a new position.

6. The method according to claim 1, wherein
a. at least one remaining region (8) having no cell (1) is removed and/or
b. a reagent is introduced into the region (2) having in particular a cell or a particle and/or
c. the region (2) having in particular a cell or a particle is at least partially sucked out or filled up.

7. The method according to claim 1, wherein the region (2) is divided into at least two sub-regions (9) by displacing the first fluid (4) of the region (2) in such a way that part of the second fluid (5) of the region (2) wets the carrier surface (7).

8. The method according to claim 1, wherein the first fluid (4) and the second fluid (5) are displaced via a displacement agent (6).

9. The method according to claim 8, wherein the displacement agent (6) and/or the carrier (3) are moved in order to displace the first fluid (4) and/or the second fluid (5).

10. The method according to claim 8, wherein
a. the displacement agent (6) has a solid body, wherein the first fluid (4) is displaced by the solid body or
b. the first fluid (4) is displaced by applying a gas from the displacement agent (6) to the first fluid (4).

11. The method according to claim 8, wherein
a. to produce the region (2) the displacement agent (6) is moved exclusively in the direction of the carrier surface (7) or the carrier (3) is moved exclusively in the direction of the displacement agent (6) or
b. to produce the region (2) the displacement agent (6) and/or the carrier (3) is moved in at least one direction parallel to the carrier surface (7).

12. A device (10) for carrying out the method according to claim 1.

13. A device (10) for producing at least one closed region (2) on a carrier surface (7) of a carrier (3) for receiving a first fluid (4) and a second fluid (5), wherein the second fluid (5) is immiscible with the first fluid (4) and at least partially covers the first fluid (4), having an optical acquisition device (25) for acquiring at least one item of cell information and/or of at least one item of particle information and a displacement agent (6), by means of which, on the basis of the at least one acquired item of cell information and/or the at least one acquired item of particle information, the closed region (2) can be produced.

14. The device according to claim 13, wherein the displacement agent (6) is designed and intended to displace the first fluid (4) in order to produce the region (2), so that part of the second fluid (15) wets the carrier surface (7).

15. The device (10) according to claim 13, wherein
a. the acquisition device (25) has an imaging device (11) for producing an image of the carrier surface (7) or
b. the acquisition device (25) has an imaging device (11) for producing an image of the carrier surface (7) and an evaluation device (12), which determines the item of cell information and/or item of particle information on the basis of the image.

16. The device (10) according to claim 15, wherein the device (10) has a receptacle (13) for receiving the carrier (3), wherein the imaging device (11) and the displacement agent (6) are mutually opposite one another with respect to the receptacle (13).

17. The device (10) according to claim 13, wherein
a. the displacement agent (6) has a hydrophobic solid body and/or
b. the displacement agent (6) has a pattern element (27) at its end which can be brought into contact with the first fluid (4) and/or second fluid (15) and/or
c. the displacement agent (6) has a gas outlet opening.

18. The device (10) according to claim 13, wherein
a. a removal device (14) for removing the remaining region (8) of the first fluid (4) having no cells and/or for removing the region (2) or
b. a removal device (14) for at least partially aspirating the remaining region (8) of the first fluid (4) having no cells and/or for at least partially sucking out the region (2).

19. A system having a device (10) according to claim 13 and a carrier (3), which receives the first fluid (4) and the second fluid (5), wherein the second fluid (5) is immiscible with the first fluid (4) and at least partially covers the first fluid (4).
